SEQUENCE LISTING (23 sequences)

SEQ ID NO: 1 - DNA, 1392 bp, Artificial Sequence
  Chemically Synthesized
gacatcgagctcacccagtctccagcctccctttctgcgtctgtgggagaaactgtcaccatcacatgtcgagcaagtgggaatattcacaattatttagcatggtatcagcagaaacagggaaaatctcctcagctcctggtctattatacaacaaccttagcagatggtgtgccatcaaggttcagtggcagtggatcaggaacacaatattctctcaagatcaacagcctgcaacctgaagattttgggagttattactgtcaacatttttggagtactcctcggacgttcggtggagggaccaagctcgagctgaagcgcgctgatgctgcaccgactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtctggtggtggttctggtggcggctctgagggtggtggctctgagggtggcggttctgagggtggcggctctgagggaggcggttccggtggtggctctggtgaagttaaactgcaggagtcaggacctggcctggtggcgccctcacagagcctgtccatcacatgcaccgtctcagggttctcattaaccggctatggtgtaaactgggttcgccagcctccaggaaagggtctggagtggctgggaatgatttggggtgatggaaacacagactataattcagctctcaaatccagactgagcatcagcaaggacaactccaagagccaagttttcttaaaaatgaacagtctgcacactgatgacacagccaggtactactgtgccagagagagagattataggcttgactactggggccaagggaccacggtcaccgtctcctcagcaaagaccactcctccgtctgtttaccctctggctcctggttctgcggctcagactaactctatggtgactctgggatgcctggtcaagggctatttccctgagccagtgacagtgacctggaactctggatccctgtccagcggtgtgcacaccttcccagctgtcctgcaatctgacctctacactctgagcagctcagtgactgtcccctccagcacctggcccagcgagaccgtcacctgcaacgttgcccacccggcttctagcaccaaagttgacaagaaaatcgtaccgcgcgat

SEQ ID NO: 2 - protein, 464 aa, Artificial Sequence
  Chemically Synthesized
DIELTQSPASLSASVGETVTITCRASGNIHNYLAWYQQKQGKSPQLLVYYTTTLADGVPSRFSGSGSGTQYSLKINSLQPEDFGSYYCQHFWSTPRTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNESGGGSGGGSEGGGSEGGGSEGGGSEGGGSGGGSGEVKLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRD

SEQ ID NO: 3 - DNA, 102 bp, Artificial Sequence
  Chemically Synthesized
tctggtggtggttctggtggcggctctgagggtggtggctctgagggtggcggttctgagggtggcggctctgagggaggcggttccggtggtggctctggt

SEQ ID NO: 4 - protein, 34 aa, Artificial Sequence
  Chemically Synthesized
SGGGSGGGSEGGGSEGGGSEGGGSEGGGSGGGSG

SEQ ID NO: 5 - DNA, 96 bp, Artificial Sequence
  Chemically Synthesized
tctggtggtggttctggtggcggctctgagggtggtggctctgagggtggcggttctgagggtggcggctctgagggaggcggttccggtggtggc

SEQ ID NO: 6 - protein, 32 aa, Artificial Sequence
  Chemically Synthesized
SGGGSGGGSEGGGSEGGGSEGGGSEGGGSGGG

SEQ ID NO: 7 - DNA, 108 bp, Artificial Sequence
  Chemically Synthesized
tctggtggtggttctggtggcggctctgagggtggtggctctgagggtggcggttctgagggtggcggctctgagggaggcggttccggtggtggctctggtggatcc

SEQ ID NO: 8 - protein, 36 aa, Artificial Sequence
  Chemically Synthesized
SGGGSGGGSEGGGSEGGGSEGGGSEGGGSGGGSGGS

SEQ ID NO: 9 - DNA, 5549 bp, Artificial Sequence
  Chemically Synthesized
tggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggtatcacgaggccctttcgtcttcacctcgagagcgggcagtgagcgcaacgcaattaatgtgagttagctcactcattaggcaccccaggctttacactttatgctcccggctcgtatgttgtgtggaattgtgagcggataacaatttcacacagaattcattaaagaggagaaattaaccatgaaatacctattgcctacggcagccgctggcttgctgctgctagcagctcagccggcgatggccgacatcgagctcacccagtctccagcctccctttctgcgtctgtgggagaaactgtcaccatcacatgtcgagcaagtgggaatattcacaattatttagcatggtatcagcagaaacagggaaaatctcctcagctcctggtctattatacaacaaccttagcagatggtgtgccatcaaggttcagtggcagtggatcaggaacacaatattctctcaagatcaacagcctgcaacctgaagattttgggagttattactgtcaacatttttggagtactcctcggacgttcggtggagggaccaagctcgagctgaagcgcgctgatgctgcaccgactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttctggtggtggttctggtggcggctctgagggtggtggctctgagggtggcggttctgagggtggcggctctgagggaggcggttccggtggtggctctggtgaagttaaactgcaggagtcaggacctggcctggtggcgccctcacagagcctgtccatcacatgcaccgtctcagggttctcattaaccggctatggtgtaaactgggttcgccagcctccaggaaagggtctggagtggctgggaatgatttggggtgatggaaacacagactataattcagctctcaaatccagactgagcatcagcaaggacaactccaagagccaagttttcttaaaaatgaacagtctgcacactgatgacacagccaggtactactgtgccagagagagagattataggcttgactactggggccaagggaccacggtcaccgtctcctcagcaaagaccactcctccgtctgtttaccctctggctcctggttctgcggctcagactaactctatggtgactctgggatgcctggtcaagggctatttccctgagccagtgacagtgacctggaactctggatccctgtccagcggtgtgcacaccttcccagctgtcctgcaatctgacctctacactctgagcagctcagtgactgtcccctccagcacctggcccagcgagaccgtcacctgcaacgttgcccacccggcttctagcaccaaagttgacaagaaaatcgtaccgcgcgattgcgcggccgcttggagccacccgcagttcgagaaatagggatccaaagatatcagagctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatgactctagaggcctgtgctaatgatcacctagcttgaggcatcaataaaacgaaaggctcagtcgaaagactgggcctttcattttatctgttgtttgtcggttaacgtcgacc

SEQ ID NO: 10 - protein, 22 aa, Artificial Sequence
  Chemically Synthesized
MKYLLPTAAAGLLLLAAQPAMA

SEQ ID NO: 11 - protein, 107 aa, Artificial Sequence
  Chemically Synthesized
DIELTQSPASLSASVGETVTITCRASGNIHNYLAWYQQKQGKSPQLLVYYTTTLADGVPSRFSGSGSGTQYSLKINSLQPEDFGSYYCQHFWSTPRTFGGGTKLELK

SEQ ID NO: 12 - protein, 107 aa, Artificial Sequence
  Chemically Synthesized
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 34 aa, Artificial Sequence; identical to SEQ ID NO: 4
  Chemically Synthesized

SEQ ID NO: 14 - protein, 116 aa, Artificial Sequence
  Chemically Synthesized
EVKLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTTVTVSS

SEQ ID NO: 15 - protein, 102 aa, Artificial Sequence
  Chemically Synthesized
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 16 - protein, 412 aa, Artificial Sequence
  Chemically Synthesized
GSKDIRAETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 17 - DNA, 1458 bp, Artificial Sequence
  Chemically Synthesized
atgaaatacctattgcctacggcagccgctggcttgctgctgctagcagctcagccggcgatggccgacatcgagctcacccagtctccagcctccctttctgcgtctgtgggagaaactgtcaccatcacatgtcgagcaagtgggaatattcacaattatttagcatggtatcagcagaaacagggaaaatctcctcagctcctggtctattatacaacaaccttagcagatggtgtgccatcaaggttcagtggcagtggatcaggaacacaatattctctcaagatcaacagcctgcaacctgaagattttgggagttattactgtcaacatttttggagtactcctcggacgttcggtggagggaccaagctcgagctgaagcgcgctgatgctgcaccgactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtctggtggtggttctggtggcggctctgagggtggtggctctgagggtggcggttctgagggtggcggctctgagggaggcggttccggtggtggctctggtgaagttaaactgcaggagtcaggacctggcctggtggcgccctcacagagcctgtccatcacatgcaccgtctcagggttctcattaaccggctatggtgtaaactgggttcgccagcctccaggaaagggtctggagtggctgggaatgatttggggtgatggaaacacagactataattcagctctcaaatccagactgagcatcagcaaggacaactccaagagccaagttttcttaaaaatgaacagtctgcacactgatgacacagccaggtactactgtgccagagagagagattataggcttgactactggggccaagggaccacggtcaccgtctcctcagcaaagaccactcctccgtctgtttaccctctggctcctggttctgcggctcagactaactctatggtgactctgggatgcctggtcaagggctatttccctgagccagtgacagtgacctggaactctggatccctgtccagcggtgtgcacaccttcccagctgtcctgcaatctgacctctacactctgagcagctcagtgactgtcccctccagcacctggcccagcgagaccgtcacctgcaacgttgcccacccggcttctagcaccaaagttgacaagaaaatcgtaccgcgcgat

SEQ ID NO: 18 - protein, 22 aa, Artificial Sequence
  Chemically Synthesized
MKYLLPTAAAGLLLLAAQPAMA

SEQ ID NO: 19 - protein, 107 aa, Artificial Sequence; identical to SEQ ID NO: 11
  Chemically Synthesized

SEQ ID NO: 20 - protein, 106 aa, Artificial Sequence
  Chemically Synthesized
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 21 - protein, 34 aa, Artificial Sequence; identical to SEQ ID NO: 4
  Chemically Synthesized

SEQ ID NO: 22 - protein, 111 aa, Artificial Sequence
  Chemically Synthesized
EVKLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTT

SEQ ID NO: 23 - protein, 106 aa, Artificial Sequence
  Chemically Synthesized
VTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRD

(19) United States
(12) Patent Application Publication
(10) Pub. No.: US 2007/0274985 A1
(43) Pub. Date: Nov. 29, 2007

(54) Antibody

(76) Inventors: Stefan Dubel, Braunschweig (DE); Martina Inga Kirsch, Leimen (DE); Michael Hust, Hannover (DE); Thomas Jostock, Neuenburg am Rhein (DE); Doris Meier, Sankt Andreasberg (DE)

Correspondence Address: WILLIAM COLLARD;COLLARD & ROE, P.C., 1077 NORTHERN BOULEVARD, ROSLYN, NY, 11576, US

(21) Appl. No.: 11/442,470

(22) Filed: May 26, 2006

(51) Int. Cl. A61K 39/395; C07K 16/18

(52) U.S. Cl. 424/133.1; 530/388.1

(57) ABSTRACT

The present invention refers to synthetic antibody molecules which comprise domains from naturally occuring antibodies, e.g. domains derivable from IgG, preferably of human origin, in a novel arrangement. Single chain molecules are provided which are suitable for expression in micro-organisms in their active conformation, which single chain molecules generally comprise a VL domain, a CL domain, and a VH domain, a CH1 domain, linked by a linker arranged between VUCL and VH/CH1. Accordingly, these antibody molecules can be termed single chain Fabs (scFabs). These antibody molecules are single chain proteins, which can also be associated to dimers, including heteromeric antibodies, wherein at least two single chain antibody molecules are associated.

The present invention relates to proteinaceous molecules having specificity to an antigen, i.e. to protein having antigen binding specificity. In greater detail, the present invention relates to a synthetic antibody comprising a single chain peptide, which can fold into a synthetic antibody having one antigen binding site, or which single chain molecule can associate with at least one further single chain synthetic antibody molecule having the same or different antigen-specific domains, to form an at least bivalent synthetic antibody molecule. In accordance with the antigen binding domains, the bivalency can refer to the same or different antigen specificities

### STATE OF THE ART

Kufer et al. (Trends in Biotechnology, 238-244 (2004)) give an overview on synthetic antibodies that have been assembled from single domains of natural immunoglobulins, e.g. IgG antibody. In natural antibodies, antigen binding takes place via associated VH and VL regions. Accordingly, one synthetic antibody fragment is a single chain (sc) FV, consisting of a VH domain and a VL domain which are connected by a peptide linker.

As used throughout the description of this invention, amino acid sequences as well as arrangements of peptide elements and protein regions, are given in from N-terminus to C-terminus.

This scFv is also termed minibody as it is the smallest antigen binding antibody with an antigen binding surface that is formed of two domains only, namely a VH and a VL domain. A known variation to the scFv is the hetero-dimeric scFv, consisting of two associated single chain polypeptides, each comprising a VH, VL domain, respectively, allowing the association of the VH and VL domains contained in different single chain peptides for forming a heterodimer. Each of the single chains has a VH domain and a VL domain with a different antigen specificity, but the antigen specificity of the VH domain of one single chain corresponds to the antigen specificity of the VL domain of the second single chain, and vice versa. Accordingly, a bivalent synthetic antibody is formed.

As a further variation of the minibody, concurrent synthesis of different single chain constructs yields a hetero-dimeric bivalent antibody, in the art also termed diabody. Therein, a first single chain consists of a VL domain with a first antigen specificity and a VH domain with a second antigen specificity, and the second single chain consists of a VH domain with the first antigen specificity and a VL domain with the second antigen-specificity. These two single chain constructs are said to be associated such that the VL domain of the first construct associates with the VH domain of the second construct, both having the same first antigen specificity, whereas the VH domain of the first construct associates with the VL domain of the second construct, both having the same second antigen specificity that differs from the first antigen specificity. As a result, a bivalent antibody is obtained, forming antigen binding surfaces specific for a first and a second antigen by the association of a VH domain and a VL domain, respectively, one domain contained in each single chain construct.

As a further development of the minibody, the tandem scFv is known, consisting of a VL and a VH domain, both having a first antigen specificity, linked in a single chain to a second VH domain and a second VL domain, both having a second antigen specificity. Again, a synthetic antibody is formed, comprising two different antigen binding surfaces formed by an associated VL and VH domain having the same antigen-specificity each.

WO 95/08577 discloses the use of a diabody for medical purposes, allowing the binding of a cell surface antigen with one of its antigen binding specificities, and binding of an antigen binding surface of a natural IgG with the other of its antigen specificities. Bivalent binding by the diabody of the natural antibody to the cell surface antigen is used to direct the recruitment of the lymphocyte response or complement activation to the cell surface antigen by engaging the FC region of the natural antibody.

For efficient selection of antigen binding proteins, it is known from WO 92/01047 to produce protein comprising a specific binding pair, e.g. in the form of a Fab encoding a VH domain, a CH1 and a g3p domain, when associated with a separate polypeptide comprised of a VL and a CL domain, or in the form of an scFv, consisting of a VH domain, a VL domain and a g3p domain, in functional form at the bacteriophage surface. The coding sequence of the fusion protein is encoded by the phagemid. The system, also known as phage display is suitable for selecting antibody constructs from a large number of mutants, allowing to isolate the nucleic acid sequence encoding the polypeptide displayed on the phage surface after a selection process, e.g. using the affinity binding to a desired antigen.

U.S. Pat. No. 6,207,804 discloses the modification of CDRs contained in synthetic antibodies, consisting of a VH domain and a VL domain, which domains can be associated non-covalently, associated by attachment to a linker peptide, or be dissociated, i.e. be present as individual polypeptides. When expressing a single chain protein containing the complete VH and VL domains with a 15 amino acid linker between them in E. coli, inclusion bodies were found, isolated and refolded.

U.S. Pat. No. 5,091,513 also focusses on the manipulation of antigen binding regions and as an example for a single chain antibody describes the synthesis of a protein consisting of a VH domain, a linker and a VL domain. When expressed in E. coli, the antibody is found in the form of inclusion bodies, i.e. in a denatured inactive conformation that requires refolding.

When producing antibodies known from the state of art having a larger molecular weight than scFv in micro-organism host cells, e.g. in E. coli, it has been found that inclusion bodies are formed, which require laborious processing steps for folding into a conformation having the desired antigen specificity, i.e. into a native conformation. To-date, expression of larger synthetic antibodies, e.g. IgG is therefore usually performed in mammalian cell culture.

A further disadvantage of known antibodies is that their relatively small molecular size that allows rapid clearing from the human body by the kidney, as it is e.g. the case for minibodies.

### OBJECTS OF THE INVENTION

In view of the shortcomings of known antibody constructs, it is an object of the present invention to provide antibodies which are suitable for production in E. coli, with at least a fraction of the antibodies produced in its native conformation, i.e. having antigen specificity.

Preferably, the present invention aims at providing antibodies, which can be expressed in micro-organisms, e.g. in, bacteria, and e.g. transported into the periplasmic space or secreted into the culture medium. It is furthermore preferred that antibody is provided which can be expressed at least in a fraction of the amount of translation product synthesized in its native conformation in eucaryotic micro-organisms, e.g. in yeast, fungal cells, insect or mammalian cells.

Further, the present invention aims at providing an antibody having a molecular size suitable to avoid rapid clearing by the human kidney in order to provide for increased longevity after administration to a patient.

Preferably, it is an object of the present invention to provide antibodies further having an at least bivalent antigen specificity, allowing the concurrent binding of at least two different antigenic surfaces.

### SUMMARY

The present invention refers to synthetic antibody molecules which comprise domains from naturally occuring antibodies, e.g. domains derivable from IgG, preferably of human origin, in a novel arrangement. In contrast to known antibody molecules, the present invention provides single chain molecules which are suitable for expression in micro-organisms in their active conformation, the single chain molecules generally comprising, a VL domain, a CL domain, and a VH domain, a CH1 domain, linked by a linker arranged between VL/CL and VH/CH1. Accordingly, these antibody molecules can be termed single chain Fabs (scFabs). These antibody molecules are single chain proteins, which can also be associated to dimers, including heteromeric antibodies, wherein at least two single chain antibody molecules are associated. In heteromeric antibodies the variable domains contained in one of the single chain molecules differ from one another, which with respect to binding the same antigen, are complementary to the pairing antigen specificities of the variable domains in the associated single chain molecule. As an example for these bispecific antibodies, the antigen specificity of the VL domain of a first single chain molecule corresponds to the antigen specificity of the VH chain of a second single chain molecule, whereas the antigen specificity of the VH domain of the first single chain molecule corresponds to the antigen specificity of the VL domain of the second single chain molecule.

For expression of scFabs according to the invention, monocistronic or polycistronic expression cassettes can be used, e.g. in the case of di- or polycistronic expression cassettes, single chain Fabs can be encoded having the same or different antigen specificities in their variable domains. In the case of expression of heteromeric antibodies comprising scFabs according to the invention, concurrent expression of both scFabs can be employed, or expression of each scFab having a VH domain and a VL domain with different antigen specificities, respectively, can be done in separate cultures, followed by isolation and contacting these scFabs in solution for in vitro association.

### GENERAL DESCRIPTION OF THE INVENTION

The present invention attains the above-mentioned objects by providing an antibody having a structure of a single chain (sc) antigen binding fragment (Fab), comprising a VL domain coupled to a first constant domain, preferably a CL domain or a CH1 domain, which are connected by a linker to a VH domain coupled to a second constant domain, preferably a CH1 domain or a CL domain, respectively. It is preferred that the first constant domain is a CL domain when the second constant domain is a CH1 domain, and that the first constant domain is a CH1 domain when the second constant domain is a CL domain. This single chain Fab is preferably characterized in that the linker has a length of 5 to 40 amino acids, preferably of 28 to 36 amino acids, more preferably of 30 to 34 amino acids. More preferable, at least one of the cystein residues of the CH1 and/or the CL domains, which are capable of forming a disulfide bond, is deleted.

Embodiments of the invention include the following arrangements of the domains and the linker, from N-terminus to C-terminus: VL-CL-linker-VH-CH1 and VH-CH1-linker-VL-CL, forming a stable scFab antibody.

For the scFab antibodies of the invention, it is assumed that the VH domain aligns with the VL domain, forming the antigen-specific site. Accordingly, both of the VH domain and the VL domain can have the same antigen-specificity, e.g. they can be derived from the same naturally occurring antibody.

Further, it is assumed that the CH1 domain and CL domain align themselves, and it is preferred that the CH1 domain and the CL domain originate or are derived from the same natural antibody. Alignment of the CH1 domain and CL domain can also occur between two single chain proteins, forming a homodimer.

It is preferred that the domains comprised in the antibody of the invention each have an amino acid sequence identical to the corresponding elements of a synthetic, preferably of a naturally occurring antibody, e.g. an IgG, preferably of human origin. Further, it is preferred that except for the linker, the VL domain and CL domain as well as the VH domain and the CH1 domain, respectively, are not separated by interstitial linkers or additional amino acid residues, i.e. is preferred that neighbouring two domains arranged to the N-terminus and the C-terminus of the linker, respectively, are directly linked to one another. This direct linkage of the two domains arranged on the N-terminus or C-terminus of the linker, respectively, is preferably realized as found in naturally occurring antibody, e.g. in immunoglobulins, preferably of human origin, e.g. IgG.

In the alternative to the variable domains, i.e. the VL and VH domains having the same antigen-specificity, forming a single chain Fab, hetero-dimeric, hetero-trimeric or hetero polymeric antibody molecules can be generated from the scFab molecules of the invention. For generating heteromeric antibody, a first scFab having a first antigen specificity in its VL domain and a second antigen specificity in its VH domain is associated with a second scFab having a VL domain with the second antigen specificity and a VH domain having the first antigen specificity. Heteromeric antibodies comprising a first scFab of the invention in which the variable domains have a first and a second antigen-specificity and, in association, a second scFab, in which the variable domains with respect to the first scFab have exchanged antigen-specificities, can be realized in all arrangements of the sequence of domains arranged at the N-terminus and C-terminus of the linker, respectively.

As a result of an antibody comprising scFab molecules of the invention wherein the antigen specificities of the variable chains in each molecule differ, bispecific, trispecific and polyspecific heteromeric antibodies are generated.

Embodiments of scFab which can associate into dimers and polymers, include but are not limited to the following single chain proteins, from N-terminus to C-terminus:

VH-CH1-linker-VL-CL, associated into homodimers and/or homopolymers, wherein the VH and VL domains have the same antigen specificity or, alternatively, wherein the VH and VL domains have different antigen specificities;

VL-CL-linker-VH-CH1 associated into homodimers and/or homopolymers, wherein the VH and VL domains have the same antigen specificity or, alternatively, wherein the VH and VL domains have different antigen specificities; and/or

a first scFab having the structure VH-CH1-linker-VL-CL associated with a second scFab having the structure VL-CL-linker-VH-CH1, in which associates the VH and VL domains have the same antigen specificity, or, alternatively, in which associates the VH and VL domains have different antigen specificities.

Antigen specificities of the variable domains can be directed against any natural or synthetic antigen, e.g. surface antigen from pathogens, e.g. of bacterial, yeast or fungal origin as well as of parasitic origin or of disease related origin, e.g. surface markers of tumor cells. Examples for tumor specific antigen are Her2/neu and CD 30.

When generating heteromeric antibody comprising scFabs of the invention having different antigen specificities in variable domains, a first antigen specificity can be directed against a pathogenic antigen, whereas the second antigen-specificity can be a lymphocyte stimulating specificity, e.g. anti-CD 89, anti-CD 16, anti-CD 3 or anti-CD 64. Bispecific heteromeric antibodies can be used to direct the cellular immune response against cells bearing the antigen of the first antigen specificity, e.g. against tumor cells or parasitic cells bearing a specific marker. The use of bispecific antibodies for therapeutic applications has been described in WO 95/08577, the disclosure of which is hereby incorporated in its entirety.

For generating VH and VL domains, respectively, having a specific antigen specificity directed against an antigen against which an immune response is desired, a variety of methods can be used. First, the respective variable domains can be derived from known antibodies having the desired antigen-specificity by analysing their amino acid sequences for incorporation into an expression vector encoding that amino acid sequence identically. Second, the variable domains having a desired antigen specificity can be selected from a pool of mutants thereof, e.g. by an expression in phage display of a pool of mutants of variable domains, followed by selecting the desired mutant by its affinity to the antigen against which antigenicity is desired. The techniques of expression in phage display including the generation of mutants are known to the skilled person, e.g. from WO 92/01047, WO 92/20791, WO 93/06213, WO 93/11236, WO 93/109172, WO 94/13804, the disclosures of which are hereby incorporated in their entirety. Further, in vitro mutagenesis of coding sequences for variable domains and their subsequent selection for a desired antigen specificity can be realized according to WO 92/01047, the disclosure of which is incorporated in its entirety.

In addition to the antigen-specificity of the antibodies of the invention, including bispecific antibodies comprising at least two scFab molecules of the invention, wherein the variable domains have differing antigen-specificities, it is a specific advantage of the antibodies according to the invention that they can be expressed in micro-organism host cells, yielding at least a fraction of synthesized protein in the native conformation. Although mammalian cell culture can be used for production of antibody according to the invention, expression in micro-organisms is preferred for production of the antibodies in their active conformation, i.e. in a conformation realizing their antigen-specificity when contacted with their specific antigen. When analyzing the expression in micro-organisms, it was found that periplasmic localization and/or secretion into the culture supernatant of antibody in its native conformation is obtained in a large variety of gram-negative and gram-positive bacterial strains, e.g. in E. coli. When using eucaryotic micro-organisms, e.g. Pichia, synthesis of active conformation antibody was obtained.

An scFab according to the invention can further be derivatized by an an amino acid sequence fused to its C-terminus, forming a single chain fusion protein comprising the biological or medical activity of the C-terminally fused amino acid sequence. The amino acid sequence can be selected from the group comprising the Fc portion of a natural immunoglobulin, e.g. a CH2 and/or a CH3 and/or a CH4 domain, a transmembrane region, a toxin, and RNase. The fusion protein can have a hinge region of a natural antibody arranged between the scFab section and the fused amino acid sequence. As an alternative to a hinge region, a linker peptide can be arranged between scFab section an fused amino acid sequence.

As a further improvement, the scFab encoding nucleic acid sequence is preceded by a coding sequence for a signal peptide, sufficient to induce periplasmic localization or secretion of the translation product.

Further, the antibodies according to the invention are suitable for phage display, which allows to efficiently generate and isolate antibodies having antigen specificity for the desired antigen.

The invention will now be described in greater detail with reference to the best mode for carrying out the invention by the following examples, which are not to be considered as a limitation of the invention.

The examples refer to the figures, wherein

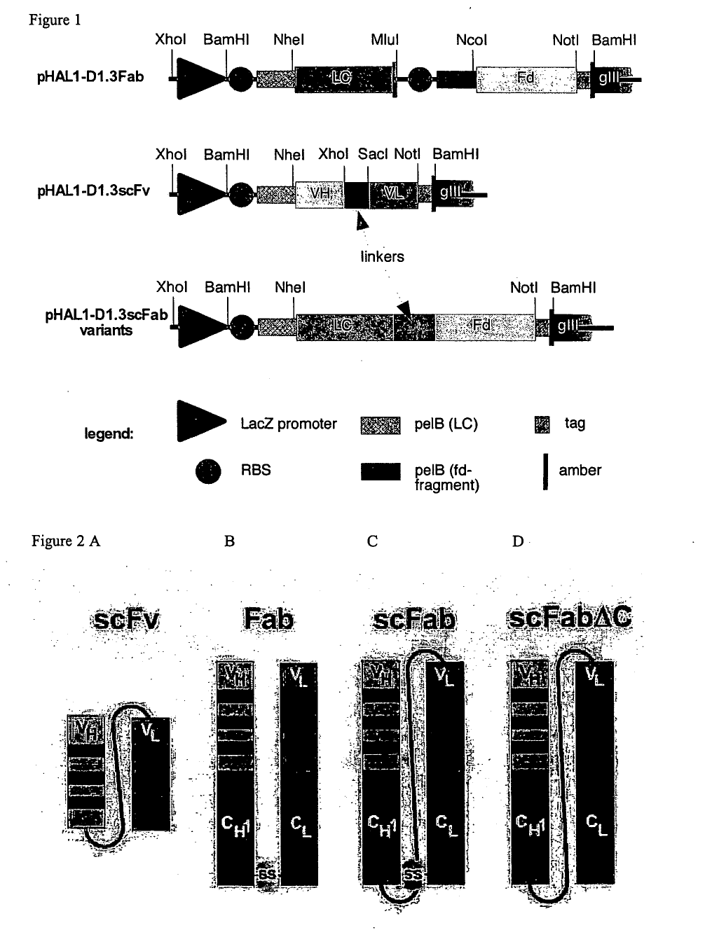

FIG. 1 schematically depicts nucleic acid constructs for expression of comparative antibodies (pHAL-D 1.3Fab, pHAL1-D 1.3scFv) and antibodies according to the invention (pHAL1-D1.3scFab variants),

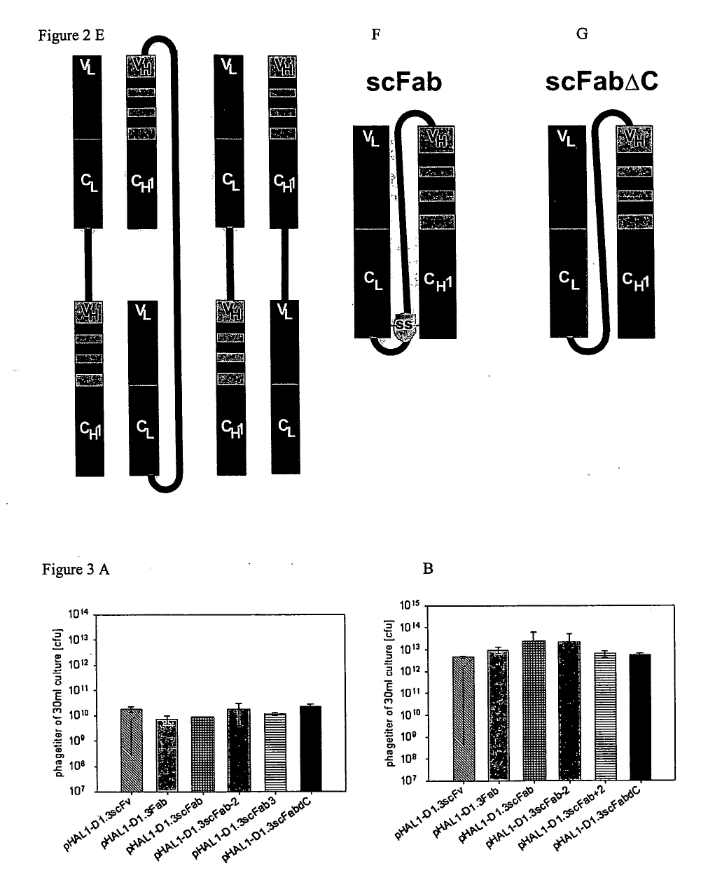

FIG. 2 schematically depicts, under A) minibody and B) Fab comparative antibodies, and under C) to G) scFabs according to the invention, including under E) the schematic representation of a dimer of scFabs of the invention,

FIG. 3 shows titers obtained for A) VCSM13, and B) for Hyperphage,

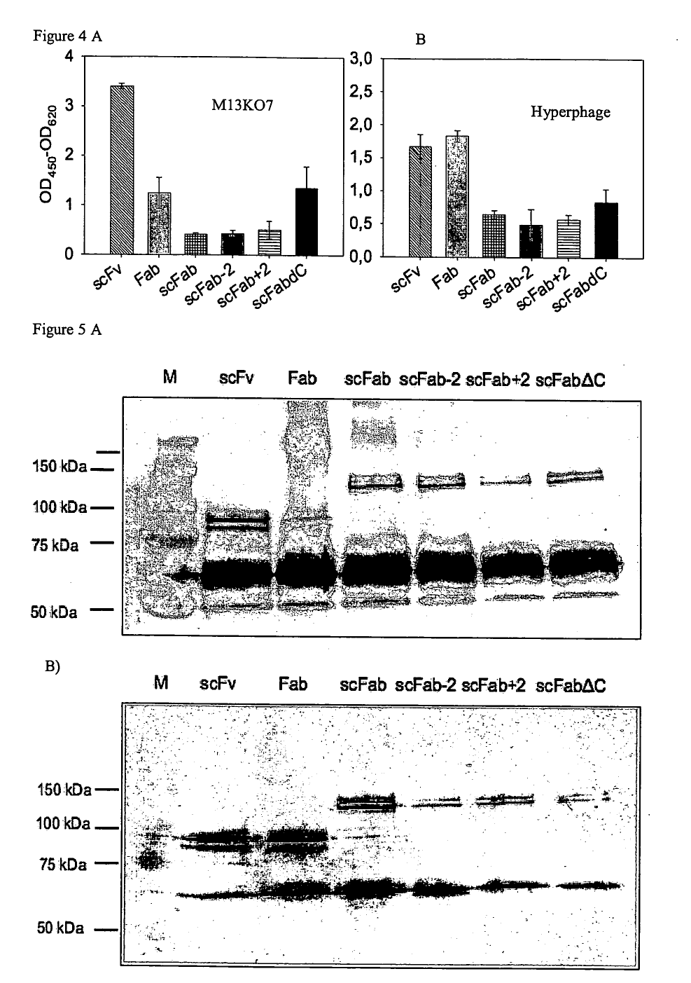

FIG. 4 shows ELISA results of phage displaying antibodies, for packaging phages A) VCSM13, and B) for Hyperphage,

FIG. 5 shows Western blots for expression of antibodies in phage display, for packaging phages A) VCSM13, and B) for Hyperphage, and

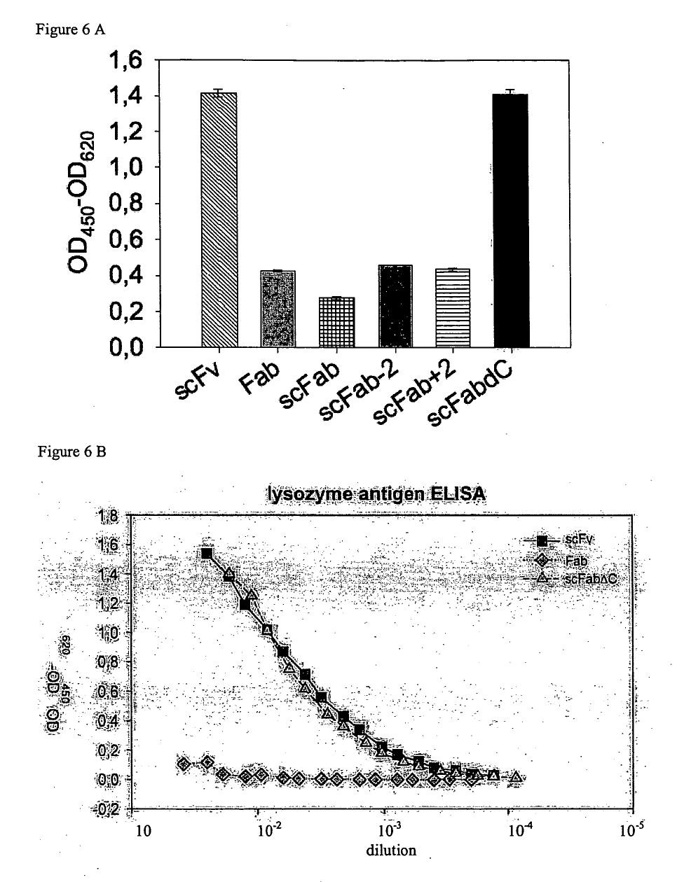

FIG. 6 shows ELISA results for antibody expression in E. coli under A), and B) from a serial dilution,

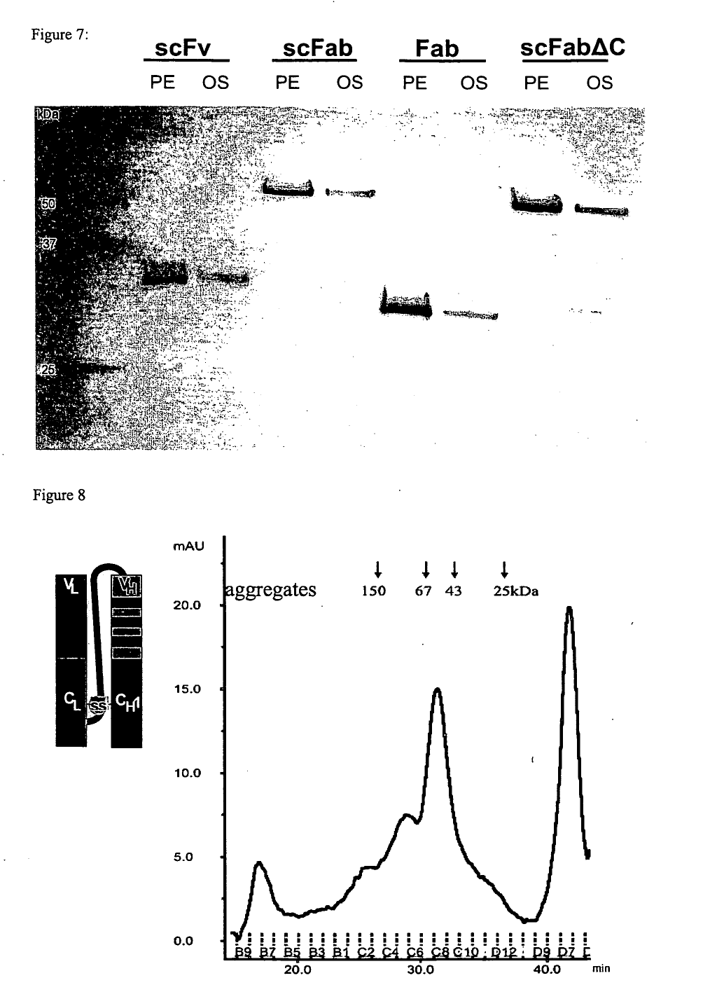

FIG. 7 shows a Western blot of the periplasmic fraction of E. coli cultures expressing the antibodies of the invention,

FIG. 8 shows a size exclusion chromatogram of an scFab of the invention having a disulfide bridge between the CL domain and the CH1 domain, isolated from the periplasm of expressing E. coli culture,

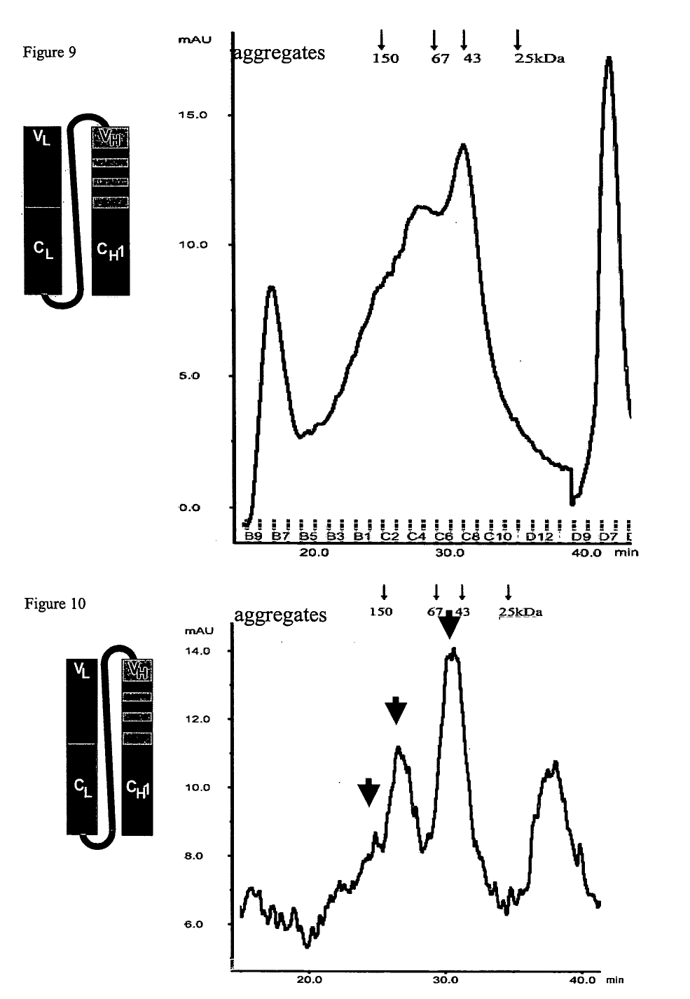

FIG. 9 shows a size exclusion chromatogram (SEC) of an scFab of the invention having no disulfide bridge between the CL domain and the CH1 domain, isolated from the periplasm of expressing E. coli culture,

FIG. 10 shows a size exclusion chromatogram of an scFab of the invention having no disulfide bridge between the CL domain and the CH1 domain, isolated from expression culture Pichia pastoris,

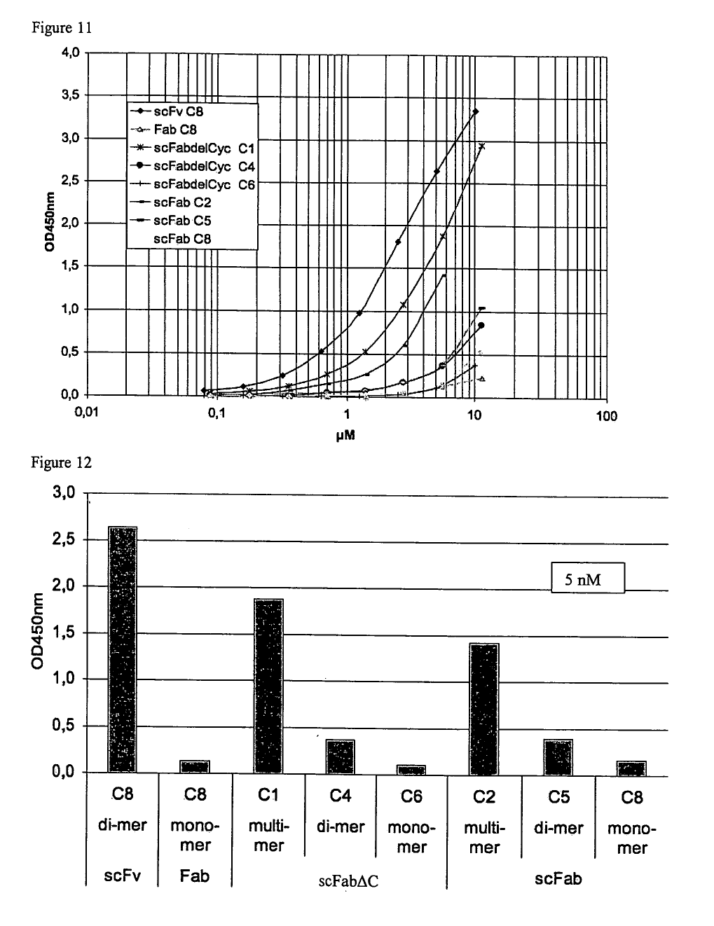

FIG. 11 shows ELISA results using increasing antibody concentrations of SEC fractions, and

FIG. 12 shows the ELISA results of antibody SEC fractions at equal concentrations.

In the examples, domains from the lysozyme binding antibody D 1.3 (Ward et al., 1989) were used as an example for any naturally occurring immunoglobulin, comprising the domains that are contained in the antibody according to the invention.

### EXAMPLE 1

### Cloning of Nucleic Acid Sequences Encoding scFab

Using standard cloning procedures (Sambrook et al., Cold Spring Harbour Laboratory, 1989), expression cassettes for scFabs were cloned into a phagemid vector.

The coding sequence for the domains VH-CH1 was amplified by PCR from phagemid vector pHAL1-D 1.3 Fab (Kirsch et al., 2005), using sequence specific primers. In a separate PCR reaction, the sequence encoding the domains VL-CL was amplified from pHAL1-D 1.3 Fab, using specific primers. The PCR amplificates contained overlapping sections, allowing to generate their fusion amplificate in a third common PCR, comprising coding sequences for a VL-CL-linker-VH-CH1 fusion protein. This coding sequence was cloned into the NheI and NotII sites of pHAL1. The cloning procedure is schematically depicted in FIG. 1. The nucleic acid sequence of the VL-CL-linker-VH-CH1 amplificate is given as Seq.-ID No. 1, the amino acid sequence as Seq.-ID-No. 2.

ScFab encoding nucleic acid sequences additionally included at its 5′-end the signal peptide pIII encoding sequence that directs the translation product to the periplasm. Coding sequences for amino acid linkers are given below:
34 amino acid linker encoding sequence, comprised in scFab (Seq.-ID No. 3, the encoded amino acid sequence as Seq.-ID No. 4),
the 32 amino acid linker (scFab−2, Seq.-ID No. 5, the encoded amino acid sequence as Seq.-ID No. 6),
the 36 amino acid linker (scFab+2, Seq.-ID No. 7, the encoded amino acid sequence as Seq.-ID No. 8), and
the 34 amino acid linker (scFabΔC) in combination with the deletion of at least one of the C-terminal Cystein residues of the CL domain and/or of the CH1 domain, which cystein residues are capable of forming a sulfide bond between the CL and CH1 domains, which is an embodiment of the most preferred scFab. Seq.-ID No. 9 gives the complete sequence of the expression plasmid for an scFab (pHAL1.3scFab), including as Seq.-ID Nos. 10 to 23 the amino acid sequences of the coding regions indicated.

The nucleic acid sequences are given by way of example only and are not intended to limit the scope of the invention. Therefore, linker sequences can be changed, e.g. by exchanging amino acid residues, but it is preferred to maintain the length of linkers. The light and constant chain domains can also be exchanged for chains from different antibodies, having the same function as VH, VL, CH1 and CL, respectively. It is especially preferred to replace the VH and VL domains for VH and VL domains having an antigen specificity for a desired antigen. In detail, the variable domains VH and/or VL can be modified by replacing the CDR hypervariable regions and/or the frame work regions with respective regions from known natural or synthetic antibodies; the CDR and frame work regions are schematically indicated by horizontal stripes. Further, the light chain domains can be lambda or kappa.

XL1-Blue MRF′ E. coli (Stratagene, Amsterdam, Netherlands) were transformed with the phagemid constructs by electroporation. Cloning was verified by sequencing of phagemid vector.

The structure of expression vectors pHAL-D1.3Fab (comparative), pHAL1-D1.3scFv (comparative) and pHAL1-D1.3scFab are shown in FIG. 1. In pHAL1-D1.3scFab, encoding the scFabs of the invention, LC indicates a VL domain coupled to a CL domain, and Fd indicates a VH domain coupled to a CH1 domain. LC indicates a leader peptide sequence directing the scFabs for export into the periplasm or for secretion into the culture medium. The tag, e.g. a His-tag, is merely included for ease of detection by tag-specific antibodies. Functional elements of the expression cassette, e.g. the promoter (LacZ), stop codon (amber) and ribosomal binding site (RBS), as well as sections in addition to the essential components of the structural sequence, comprising a first variable domain coupled to a first constant domain, a linker, and a second variable domain coupled to a second constant domain, can be exchanged for elements of equal function, e.g. the optional additional leader peptide sequence or the marker tag (e.g. His-tag).

In FIG. 2, schematic representations of synthetic antibodies are shown, including A) the known scFv consisting of a VH domain, a linker and a VL domain, B) a Fab, consisting of a VH domain coupled to a CH1 domain, the latter forming a disulfide bridge to the CL domain of a VL domain coupled to a CL domain, and, according to the invention, as C) an scFab comprising a VH domain directly coupled to a CH1 domain, linked by a linker to a VL domain that is coupled to a CL domain, wherein the CH1 and CL domains form a disulfide bridge, and as F) an scFab comprising a VL domain coupled to a CL domain, followed by a linker and a VH domain coupled to a CH1 domain, wherein the constant domains form a disulfide bridge by their C-terminal cystein residues. A preferred embodiment is given as D) and G), the scFabΔC, corresponding to the scFab, except that at least one, preferably both of the C-terminal cysteine residues of the CH1 and CL domains are deleted in order to avoid the formation of a disulfide bridge. For the scFab structures of the invention, the couple of a VH and a CH1 domain as well as the couple of a VL and a CL domain can formally be exchanged for one another, leaving the linker between these couples.

The dimerization of scFabs according to the invention is schematically depicted in FIG. 2 E), showing the assumed association of a first variable (VL) domain coupled to a first constant (CL) domain of a first scFab to the second variable (VH) domain and its coupled second constant (CH1) domain of a second scFab, respectively. Accordingly, the second variable (VH) domain and its coupled second constant (CH1) domain of the first scFab associate with the first variable (VL) domain and the first constant (CL) domain of the second scFab. In the figures, the sizes of domains and linkers are not drawn to scale, especially in FIG. 2E), all linkers can have the same number of amino acids. However, it is preferred to use linkers of 5 to 27 amino acids when generating scFabs for association into dimers. In the dimers of FIG. 2E), one scFab can have variable domains with different antigen specificities, but corresponding to the antigen specificity of the associated variable domain of the other scFab. Alternatively, the antigen specificities of the variable regions of the associated scFabs can be directed against the same antigen. Associates of scFabs of the invention into dimers, trimers and polymers, having variable domains with the same or different antigen specificities, are also termed diFabody and polyFabody, respectively.

### EXAMPLE 2

### Expression of Active scFab Antibody by Phase Display

For production of antibody presenting phage, 50 mL 2× TY medium containing 100 μg/mL ampicillin and 100 μM glucose were inoculated with an overnight culture having an OD600 of about 0.025. Bacteria were incubated at 37° C. under agitation at 250 rpm to an OD₆₀₀ of about 0.4 to 0.5. Of this culture, 2 mL were infected with 2×10¹⁰ helperphage VCSM13 (Stratagene), or Hyperphage (Rondot et al., 2001), incubated for an additional 30 minutes at 37° C. without shaking, followed by 30 minutes at 250 rpm. Infected cells were harvested by centrifugation for 10 minutes at 322× g and the cell pellet was resuspended in 13 mL 2× TY, 100 μg/mL ampicillin and 50 μg/mL kanamycin, containing various glucose concentrations. Phage were produced at 30° C. at 250 rpm for 16 hours. Cells were pelleted for 10 minutes at 10,000× g. The phage in the supernatant were precipitated with one fifth volume of a 20% by weight PEG/2.5 molar sodium chloride solution for one hour on ice with gentle shaking, followed by pelleting for one hour at 10000× g at 4° C. Precipitated phage were resuspended in 10 mL phage dilution buffer (10 mM Tris, 20 mM sodium chloride, 10 mM EDTA, pH adjusted to 7.5 using HCl), followed by a second precipitation with one fifth volume PEG solution as above for 20 minutes on ice and pelleted again at 10000× g for 30 minutes at 4° C. Precipitated phage were resuspended in 300 μL phage dilution buffer and cell debris was pelleted by an additional centrifugation for 5 minutes at 15400× g at 20° C. The supernatant containing the antibody presenting phage were stored at 4° C. Phage titration for the determination of plaque forming units (PFU) was done according to Koch et al. (2000), but packaging the infected bacteria directly onto LB-agar plates, omitting nitrocellulose sheets. When using an E. coli mutator strain for amplifying the phagemid containing the coding sequence for an scFab, a large variety of random mutants could be generated. Subsequent production of phage presenting the mutant scFabs could be used for expression of these mutant antibodies. The selection of an scFab having the desired antigen specificity could be done by standard procedures, e.g. by incubation of the phage presenting the mutant scFab population with the desired antigen that was linked to an immobilizing surface. Following interaction of the phage presented scFabs with the immobilized antigen, the coding sequence could be isolated from the isolated phage after removal of unbound phage species. Preferably, consecutive rounds of incubation of the scFab mutant phage population and the desired immobilized antigen were used to select the phagemid encoding the desired scFab.

An antigen binding ELISA could be employed for both antigen displaying phage and soluble antigen by using microtiter plates (Costar, Cambridge, USA), that were coated with 100 ng D 1.3 as the model antigen in 100 μL 0.1 M sodium carbonate solution at pH 9.6 per well overnight at 4° C. After coating, wells were washed three times with PBS and blocked with 2% by weight skim milk powder in PBS for 1.5 hours at room temperature, followed by three times washing with PBS. ScFab expressing phage or periplasmic fractions from E. coli cultures expressing the scFab were incubated on the coated microtiter plates after dilution in blocking solution, followed by five times washing with PBST (PBS containing 0.1% vol./vol. Tween-20).

Detection of bound antibody presenting phage was with monoclonal anti-m13 antibody, conjugated with HRP (Amersham Biosciences), diluted 1:5,000. In the case of periplasmic supernatants, e.g. soluble scFab antibody, detection was done with a mouse anti-Strep-tag antibody (Qiagen, Hilden, Germany), in a 1:10,000 dilution, followed by goat anti-mouse mAb, conjugated with HRP (1:50,000) or with protein L conjugated with HRP (Pearce, Bonn, Germany) in a 1:10,000 dilution, followed by visualisation with TMB substrate (Biorad, Munich, Germany). The staining reaction was stopped by addition of 100 μL 1N sulfuric acid. Absorbances at 450 and 620 nm were recorded on a Sunrise microtiter plate reader (Tecan, Germany). Absorbance of scattered light at 620 nm was substracted from absorbance at 450 nm.

Using the model scFab antibody, the antigen coated onto the microtiter plates for immobilization was lysozyme. When using VCSM13 was used in phage rescue, 5×10⁶ phage per well were applied, whereas 10⁷ phage per well were employed after packaging with Hyperphage to compensate for differences in antibody presentation efficiency.

For comparison, an scFv antibody was constructed, consisting of VH and VL domains only, as well as a phagemid encoding a Fab fragment, consisting of two expression cassettes, encoding the VH domain in connection with the CH1 domain, and the VL domain coupled to the CL domain, respectively.

Different results were obtained for Hyperphage packaging, wherein the scFv and the Fab gave best binding results, whereas the scFab variants of the invention achieved values of about a third of the activity. Among the scFabs, the scFabΔC achieved the best results. Phage titers are shown in FIG. 3A for VCSM13 and in FIG. 3B for Hyperphage packaging. These results show that the scFab and antibodies of the invention yield comparable titers to the scFv and the Fab, which titers are well sufficient for utility in phage display techniques.

The results of the antigen presenting phage ELISA are shown in FIG. 4A for VCSM13 and in FIG. 4B for Hyperphage. Using the VCSM13, the scFv antibody showed outstanding antigen binding, whereas the Fab and the scFab variants only showed half of the binding capacity. Among the scFab variants of the invention, the scFabΔC showed best affinity to the antigen.

Antibody presenting phage preparations were further analysed by SDS-PAGE under reducing conditions, followed by blotting onto PVDF membrane. The pIII leader peptide was visualized by immune staining using monoclonal mouse anti-pIII antibody. In SDS-PAGE, pIII runs at an apparent molecular mass of 65 kDa, although it has a calculated molecular mass of 42.5 kDa (Goldsmith and Konigsberg 1977). The Western blot is shown in FIG. 5A for the VCSM13 packaged phage, and in FIG. 5B for the Hyperphage preparations. The result shows that monovalent phage display using the VCSM13 yields a lower amount of fusion protein, indicated by the dominating pIII band at 65 kDa. For phage generated with Hyperphage, the antibody-pIII fusion protein is more prominent, indicated by amounts almost equal to that of unfused pIII. In this analysis, comparative scFv and Fab as well as scFab antibody according to the invention show a slightly improved level of phage display in comparison to the other antibodies.

### EXAMPLE 3

### Bacterial Expression of Active scFab Antibody

For expression of scFab antibodies according to the invention, bacteria can be used as host organisms to yield active conformation scFab.

Soluble antibody was expressed in shake flasks using 2× TY medium (Sambrook et al., 1989), supplemented with 100 μg/mL ampicillin, 100 mM glucose with an inoculation of 1:20 vol./vol. with an overnight culture of the transformed XL1-Blue. Cultivation was at 37° C. at 350 rpm for 2 hours. Bacteria were harvested by centrifugation at 3,900× g for 20 minutes. The pellet was resuspended in 100 mL 2× TY medium with 100 μg/mL ampicillin and 20 μM IPTG and incubated at 30° C. at 350 rpm overnight. Following harvesting by centrifugation, the pellet was resuspended in 13 mL PBS (phosphate buffered saline, Sambrook et al. 1989), supplemented 1% Tween-20, and incubated at 30° C. at 350 rpm for a further 2.5 hours. Centrifugation for 10 minutes at 7,000× g separated cells from supernatant, which contained the antibody.

Alternatively, expression was done in microtiter plates using 200 μL 2× TY medium with 100 μg/mL ampicillin and 100 μM glucose, inoculated with 10 μL overnight culture, and incubation at 37° C. with agitation at 1,400 rpm for 2 hours. Bacteria were harvested by centrifugation for 10 minutes at 2,500× g. After resuspension of the pellet in 200 μL 2× TY with 100 μg/mL ampicillin and 20 μM IPTG at 30° C. at 1,400 rpm overnight, 50 μL PBS including 1% Tween-20 was added, followed by incubation at 30° C. and 1,400 rpm for an additional 3.5 hours. Again, cells could be separated from the antibody containing supernatant by centrifugation for 10 minutes at 3,200× g.

For production, E. coli strain XL1-Blue was transformed with pHAL1-D1.3 constructs, encoding comparative antibodies scFv and Fab, as well scFab, scFab −2, scFab +2 and scFabΔC according to the invention, respectively. After centrifugation, periplasmic fractions were analysed by antigen ELISA, using protein L for detection.

The result is shown in FIG. 6A, indicating the effective production for the comparative scFv antibody and the scFabΔC antibody according to the invention, whereas comparative Fab antibody and scFab, scFab −2 as well as scFab +2 yielded considerably lower production rates. As the ELISA is based on the affinity of the antibodies to their specific antigen, exemplified by lysozyme, these ELISA results represent the fraction of antibody synthesized in the correctly folded state, i.e. in their native conformation having affinity to the specific antigen.

FIG. 7 shows a Western blot of comparative antibodies scFv and Fab and antibodies according to the invention scFab and scFabΔC, for periplasmic (PE) and intracellular (OS) fractions, respectively. Identification of antibodies was done with an anti-His-tag antibody, as all constructs included a His-tag. The results indicate that the antibodies of the invention are predominantly expressed in the periplasm.

FIGS. 8 and 9 show size exclusion chromatograms of obtained from periplasmic fractions of E. coli, expressing scFab (FIG. 8) and scFabΔC (FIG. 9), respectively. Antibody structures are given schematically. By comparison with the indicated molecular weights, it can be derived that the single chain antibodies of the invention also associate to dimers, trimers and multimers without any further treatment or derivatisation.

This is a demonstration of the feasibility to produce antibody according to the invention in its active conformation within a bacterial host organism. Accordingly, mammalian cell culture is no prerequisite for efficiently expressing scFab according to the invention in its natural conformation, and bacterial expression can be used without subsequent denaturing and refolding of originally inactive protein that has been obtained for state of the art antibody constructs as inclusion bodies.

Similar results were obtained when expressing the scFab antibodies of the invention in Pichia, using a yeast expression cassette. FIG. 10 shows the size exclusion chromatogram of culture supernatant obtained from Pichia pastoris transformed with a yeast expression cassette encoding an scFabΔC antibody of the invention. The antibody structure is schematically shown. The positions of marker proteins are indicated, along with fat arrows, indicating monomeric scFab (right hand arrow), dimeric scFab (middle arrow) and multimeric scFab (left hand arrow).

The preferred embodiment of the present invention, namely the scFab having a 34 amino acid linker with at least one cystein within the VH domain and/or the CL domain deleted, which cysteines are capable of forming a disulfide bond, is currently termed scFabΔC. When testing serial dilutions of periplasmic E. coli supernatant containing comparative scFv antibody and scFabΔC according to the invention, an ELISA using lysozyme as the antigen could demonstrate the production of active conformation antibody. The results are shown in FIG. 6B, demonstrating that scFabΔC yields about equal concentrations of active conformation antibody as scFv, whereas the concentration of active conformation Fab was lower by a factor of about 100.

### EXAMPLE 3

### Analysis of Association of scFab

Analysis of the association of antibodies was done for comparative constructs scFv, Fab, and for antibodies of the invention, namely scFabΔC and scFab.

The antibody constructs of the invention were expressed in E. coli and isolated from the periplasmic fraction of the culture by SEC, whereas the comparative antibodies were expressed in mammalian cell culture and isolated from culture supernatant by SEC:

scFv (comparative): VH-linker-VL

Fab (comparative): VH-CH I VL-CL, connected by a disulfide bridge

scFabΔC: VL-CL-linker-VH-CH1, wherein both C-terminal cysteins of the CL and CH1 domains were deleted,

scFab: VL-CL-linker-VH-CH1,

wherein C1, C2, C5, C6, and C8 indicate SEC fractions. In accordance with SEC fractions, antibodies are designated as dimers or multimers in FIGS. 11 and 12.

The results of an ELISA as described in Example 2, using increasing concentrations of the respective antibody, are depicted in FIG. 11. The increase of absorption at 450 nm for increasing antibody concentrations indicates the increase in antigen binding valency, and hence the association of single chain antibodies to dimers and higher multimers.

The SEC fractions were further analyzed by an ELISA using the model antigen lysozyme adsorbed onto plates as described in Example 2. Results are shown in FIG. 12, demonstrating that the scFab antibodies of the invention have increased antigen binding when present as dimers or multimers.

1. A single chain protein having antibody specificity towards an antigen, comprising a first variable domain connected to a first constant domain, a linker, and a second variable domain connected to a second constant domain.

2. A single chain protein according to claim 1, wherein the first variable domain is a VL domain and the second variable domain is a VH domain.

3. A single chain protein according to claim 1, wherein the first variable domain is a VH domain and the second variable domain is a VL domain.

4. A single chain protein according to claim 1, wherein the first constant domain is a CL domain, and the second constant domain is a CH1 domain.

5. A single chain protein according to claim 1, wherein the first constant domain is a CH1 domain, and the second constant domain is a CL domain.

6. A single chain protein according to claim 1, wherein the arrangement of domains is, from N-terminus to C-terminus of the following: VL-CL-linker-VH-CH1 and VH-CH1 -linker-VL-CL.

7. A single chain protein according to claim 1, wherein the linker comprises from 5 to 40 amino acids in length.

8. A single chain protein according to claim 1, wherein at least one of the C-terminal cystein residues comprised in the first constant domain and comprised in the second constant domain is deleted.

9. A single chain protein according to claim 1, wherein the antigen specificity of the VL domain and of the VH domain are directed against the same antigen.

10. A single chain protein according to claim 1, wherein the antigen specificity of the VL domain and of the VH domain are directed against different antigens.

11. A single chain protein according to claim 1, comprising a signal peptide sequence directing the protein for secretion.

12. A single chain protein according to claim 1, having fused to its C-terminus an amino acid sequence selected from the group comprising the Fc portion of a natural immunoglobulin, a transmembrane region, a toxin, and RNase.

13. An association of single chain proteins comprising at least one first single chain protein and at least one second single chain protein, wherein the first and the second single chain proteins comprise a VL domain connected to a CL domain, linked by a linker to a VH domain connected to a CH1 domain, wherein the linker comprises from 5 to 40 amino acids in length, and wherein the VH and VL domains of each single chain protein independently have the same or different antigen specificities.

14. An association of single chain proteins comprising at least one first single chain protein and at least one second single chain protein, wherein the first single chain protein comprises a VL domain connected to a CL domain, linked by a linker to a VH domain connected to a CH1 domain, wherein the linker comprises from 5 to 40 amino acids in length, the second single chain protein comprising a VH domain connected to a CH1 domain, linked by a linker to a VL domain connected to a CL domain, wherein the linker comprises from 5 to 40 amino acids in length, wherein the VH and VL domains of each single chain protein independently have the same or different antigen specificities.

15. An association of single chain proteins comprising at least one first single chain protein and at least one second single chain protein, wherein the first and the second single chain proteins comprise a VH domain connected to a CH1 domain, linked by a linker to a VL domain connected to a CL domain, wherein the linker comprises from 5 to 40 amino acids in length, and wherein the VH and VL domains of each single chain protein independently have the same or different antigen specificities.

16. A single chain protein according to claim 1, having fused to its C-terminus an amino acid sequence comprising the Fc portion of an RNase.
